(19) United States
(12) Patent Application Publication
(10) Pub. No.: US 2025/0064570 A1
(43) Pub. Date: Feb. 27, 2025

(54) SOFT TISSUE REPAIR PROSTHESIS WITH HANDLING AIDS AND METHODS OF USE

(71) Applicant: Davol Inc., Warwick, RI (US)

(72) Inventors: Daniel F. McCormick, Bolton, MA (US); Ricky N. Gomes, North Providence, RI (US); Korel Cudmore, Acushnet, MA (US); Augustus Felix, Cranston, RI (US); Talia J. D'Ambruoso, Sunnyvale, CA (US)

(21) Appl. No.: 18/718,769

(22) PCT Filed: Dec. 12, 2022
(86) PCT No.: PCT/US2022/052493

§ 371 Date: Jun. 11, 2024

Related U.S. Application Data
(60) Provisional application No. 63/289,039, filed on Dec. 13, 2021.

(51) Int. Cl. A61F 2/00

(52) U.S. Cl. CPC A61F 2/0063

(57) ABSTRACT

An implantable prosthesis may include a substantially planar body (10) and at least one handling aid (55). The handling aid may serve as a grab point to allow an operator (e.g., surgeon) to manipulate the planar body. reducing the risk of applying an undesirable amount of stress or strain on the underlying body. The handling aid may be an embroidered jump stitch such that it may be affixed to the planar body at least at two points. with a central portion being unattached to the plane of the body to provide sufficient clearance to the operator to grasp and handle the aid. In some embodiments, the handling aid may be in the form factor of a thread-like material. a strip. a tube. and/or a pocket. The handling aid may be used in minimally invasive surgeries such as laparoscopic and/or robotic surgeries.

### CROSS-REFERENCE TO RELATED APPLICATIONS

This application claims the benefit of U.S. Provisional Application No. 63/289,039, filed Dec. 13, 2021, which is hereby incorporated by reference in its entirety.

### FIELD

Disclosed embodiments are related to implantable mesh prostheses and related methods of use. More specifically, methods and apparatuses related to mesh prostheses with handling aids are disclosed.

### BACKGROUND

One technique for repairing a soft tissue defect involves positioning an implantable soft tissue prosthesis, such as a mesh, to cover the tissue defect. The prosthesis may be secured in place relative to the defect with tacks, sutures, and/or adhesives.

A hernia defect is an opening or weakness in a tissue or muscle wall, such as the abdominal wall. One approach for repairing a hernia is to insert a soft tissue prosthesis into an intra-abdominal space, positioning the prosthesis to cover the wall defect, and then, if desired, securing the prosthesis to the abdominal wall with tacks, sutures, and/or adhesives.

For repairs of various different soft tissue defects, a prosthesis may be delivered through a minimally invasive technique, such as a laparoscopic procedure. For example, to deliver the prosthesis intra-abdominally, the prosthesis may be rolled up, folded or otherwise collapsed into a reduced configuration and then inserted through a small incision or a trocar and into the intra-abdominal space. The prosthesis is then unfurled and positioned relative to the defect.

### SUMMARY

In some embodiments, surgical prostheses are provided. A surgical prosthesis may include a mesh comprising a first face and a second face and a loop distinct from the mesh. The loop may be attached to at least two locations on the mesh. The loop may have a central portion between the at least two locations, the central portion being disposed on a first side of the first face.

In some embodiments, methods of handling surgical prostheses are provided. A method of handling a surgical prosthesis may include grasping a central portion of a first loop, which may be attached to two locations on a mesh of the prosthesis, and manipulating a position of the central portion with respect to a defect in a patient. The mesh may include a first face and a second face. The first loop may be distinct from the mesh.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 schematically illustrates a mesh prosthesis with handling aids according to some embodiments;

FIGS. 2A-2B schematically illustrate a mesh prosthesis with handling aids according to some embodiments;

FIG. 3 schematically illustrates a portion of a mesh prosthesis with handling aids according to some embodiments;

FIG. 4 shows a perspective top view of a mesh prosthesis with handling aids according to some embodiments;

FIG. 5 shows a perspective top view of a mesh prosthesis with handling aids in operation according to some embodiments;

FIG. 6 shows a top plan view of a mesh prosthesis with handling aids according to some embodiments;

FIG. 7 schematically illustrates a perspective top view of a mesh prosthesis with handling aids according to some embodiments;

FIG. 8 shows a mesh prosthesis with handling aids in operation according to some embodiments;

FIGS. 9A-9B schematically illustrate mesh prostheses with handling aids according to some embodiments;

FIG. 10 schematically illustrates a mesh prosthesis with handling aids according to some embodiments; and

FIGS. 11A-11C show a process of preparation of a mesh prosthesis with handling aids according to some embodiments.

### DETAILED DESCRIPTION

In conventional laparoscopic soft tissue repair procedures, surgeons often place an implantable prosthesis in an internal biological space or pocket of a subject (e.g., patient) to provide support and/or strength to the weakened tissue and expedite the repair process. For example, in a hernia repair procedure, the surgeon may place the prosthesis in the extraperitoneal plane. The prosthesis can be formed of a mesh-like sheet, slightly larger than the defect. The high surface area of the porous prosthesis can induce tissue ingrowth during the repair process. The prosthesis is typically sufficiently flexible to conform to the curved surfaces of the soft tissue and move along with the subject, reducing the risk of discomfort.

A surgeon and/or operator may manipulate the prosthesis within the internal biological space or pocket with one or more surgical tools and/or by directing a robotic end effector inserted into the surgical site. The prosthesis can be grasped and moved or oriented relative to the defect. The inventors have recognized that grasping the prosthesis to manipulate it may result in damage of the prosthesis. In cases where the prosthesis is formed of a mesh-like structure, excess strain from grasping or re-orienting may result in undesirable alterations of the underlying mesh structure, which may reduce the effectiveness of the mesh for defect repair.

Based on the foregoing, the inventors have recognized the benefits associated with an implantable prosthesis with one or more features to improve the handling (e.g., transportation, positioning, orienting, etc.) of the prosthesis during a procedure (e.g., within a surgical pocket, such as an extraperitoneal plane for hernia repair procedures). The one or more features may be coupled to the main body of the prosthesis, which may serve as structural support for the soft tissue defect, such that the prosthesis may be manipulated through handling of the one or more features. The features may therefore serve as grab points of the prosthesis, allowing an operator to manipulate the prosthesis body by handling the features instead of the prosthesis body (e.g., a face or corner of the prosthesis). In this way, the risk of damaging the structural integrity of the prosthesis body may be reduced. The inventors have also recognized the benefits associated with one or more handling features which do not significantly change the structure (e.g., shape, size, strength) of prosthesis body, such that the prosthesis with handling features may be used in a conventional laparoscopic and/or robotic surgery.

In some embodiments, an implantable prosthesis may include a prosthesis body and one or more handling aids. The body may be substantially planar and flexible in order to conform to one or more soft tissue sites. In some embodiments, the flexibility of the body may allow an operator (e.g., surgeon and/or surgical tool) to roll the prosthesis without appreciable loss of structural integrity and deliver said prosthesis to a surgical site (e.g., through a port and/or trocar).

The handling aids may be attached to the prosthesis body such that the body may be manipulated through handling (e.g., grasping, moving, orienting, etc.) of the handling aids. The handling aids may include one or more portions unattached to the prosthesis body, providing a clearance under which an operator may grasp the handling aids and manipulate the prosthesis. The structure and arrangement of the handling aids may be designed such that the overall footprint of the prosthesis, as well as the mechanical properties, may not be substantially changed due to the presence of handling aids. In other words, a prosthesis with handling aids of the present disclosure may be integrated into conventional laparoscopic and/or robotic surgeries and associated tools. For example, the prosthesis with handling aids may be delivered to a surgical site through a conventional 8 mm port with a similar insertion force of a prosthesis without handling aids.

The substantially planar prosthesis body may have a first side which may be positioned against a tissue or muscle wall (e.g., an abdominal wall) including the defect, as well as a second side, which may be located adjacent to sensitive nearby organs (e.g., intestines and/or other viscera). As such, the first side, which may be referred to in some embodiments as the anterior side, may be configured for tissue ingrowth. In some embodiments, the anterior side may be rough and macroporous, with an increased surface arca which may induce tissue ingrowth during a soft tissue repair procedure (e.g., hernia repair)-expediting the repair process. The second side, which may be referred to in some embodiments as the visceral side, may be configured to reduce the risk of adhesions forming with the visceral organs. As such, the visceral side may be smoother than the anterior side, and may include one or more anti-adhesion barrier layers.

In a minimally invasive technique, as well as in certain open procedures, an implantable prosthesis may be reduced in size to fit a defect and/or facilitate delivery of the prosthesis to the surgical site. For example, in a laparoscopic procedure, the implantable prosthesis may be rolled into a slender cylindrical shape, or otherwise collapsed into a smaller configuration, suitable for passage through a narrow cannula which may have an inner diameter of approximately 10 mm, of approximately 5 mm, or even a finer size. To assist in unfurling the prosthesis into an expanded shape after deployment through a cannula, an operator may grasp one or more handling aids and guide the prosthesis (which may have a propensity to return to its planar structure) towards a pocket in the surgical site. The handling aids may be grasped to manipulate, orient, and/or position the body at the defect site, with a reduced risk of damage to the underlying prosthesis due to grasping. The prosthesis may subsequently be fixed against the soft tissue repair site with any suitable means (e.g., barbs, sutures, tacks, staples, etc.).

In some embodiments, a handling aid may be a thread-like material spanning more than one pore of a prosthesis body. The thread-like material may be fixed to at least one point on the prosthesis body, such that an operator may grasp the unattached portion of the handling aid to manipulate the prosthesis body. In some embodiments, a handling aid may be fixed to the prosthesis body at two points, and the thread-like material between the two points may be unattached to the plane of the body. In some embodiments, the thread-like material between the two points may be sufficiently spaced from the plane of the body to allow an operator and/or tool to grasp said handling aid. In some embodiments, a handling aid may be formed as a closed loop normal to the plane of the prosthesis body. In some embodiments, the thread-like handling aid may be attached to the underlying prosthesis body (e.g., mesh), such that the handling aid may be distinct from the prosthesis.

The thread-like material of the handling aid may not significantly alter the strength, weight, and/or volumetric footprint of the prosthesis body, such that the prosthesis may be used with conventional tissue repair techniques. In some embodiments, the handling aid may be low-profile and present no notable impact on the insertion force required to deliver the prosthesis through one or more ports and/or trocars.

In some embodiments, a handling aid may be embroidered or sewn on to the prosthesis body. In some embodiments, the closed-loop form factor of the handling aid—which may serve as the grab point of the prosthesis body-may be achieved with a jump stitch. A jump stitch is a connective stitch in between two stitches. The jump stitch may have a longer stitch length (measured along the stitch line) than the two stitches connected by the jump stitch. As a result, a clearance may be formed in between the jump stitch and the underlying substrate. In other words, the jump stitch may have a portion that is unattached to the underlying substrate in between the two stitches connected by the jump stitch. Of course, embodiments of a jump stitch without any neighboring stitch lines are also contemplated, as described in further detail below.

The handling aid may be accessible from one or more sides of the prosthesis body, such that an operator may grasp the prosthesis from a first side (e.g., a visceral side, e.g., during unfurling of the prosthesis) and/or from a second side (e.g., an anterior side, e.g., during prosthesis orientation and positioning relative to the defect).

In some embodiments, the handling aid may be in-line with one or more embroidered and/or stitched lines on the prosthesis body, which may serve as visual and/or tactile indicia for the operator. The indicia, which may facilitate sizing, positioning, and/or orienting of the handling aids during operation, may include one or more axial lines, spatial markers, and/or spatial identifiers, as will be described in further detail below. In some embodiments, the handling aid may be positioned independently of any indicia, the handling aid itself being formed of a material distinct from the underlying prosthesis body. In some embodiments, the handling aid may itself serve as an indicator.

In some embodiments, the indicia may be visually and/or optically apparent to an operator prior to installation, so that the operator may size, shape, and/or handle the body in a suitable manner. In some embodiments, the indicia and/or handling aids may be visible to a laparoscopic camera or any other suitable surgical tool. As such, the indicia and/or handling aids may include one or more properties which may be distinct from the prosthesis body. For example, the indicia and/or handling aids may be embroidered on the body with a thread-like material. In some embodiments, the indicia and/or handling aids may be colored differently from the prosthesis body. The indicia and/or handling aids may also include one or more orienting features which may assist the operator in orienting the body. For example, the spatial identifiers may include letters or numbers visible from both faces of the body, which may distinguish one face of the body from another. If the body is oriented with one side facing the operator, based on the handedness or orientation of the spatial identifiers, the operator may be able to determine which side of the prosthesis body he or she is facing. Such a functionality may be useful for embodiments of prostheses in which the body may include one or more features (e.g., roughness, anti-adhesion coatings, etc.) on at least one side (e.g., visceral side, anterior side) designed to induce a particular biological reaction at an installation site.

In some embodiments, the indicia and/or handling aids may be colored, such that the indicia and/or handling aids may be visually distinct from the underlying prosthesis body. The indicia and/or handling aids may be colored with any suitable biocompatible color to render the indicia and/or handling aids any suitable color, including, but not limited to, blue, green, black, purple, indigo, and/or any other color. The prosthesis may include multiple indicia and/or handling aids colored with more than one color, to differentiate the indicia and/or handling aids from one another. In some embodiments, one or more indicia and/or handling aids may be transparent in color. In some embodiments, the indicia and/or handling aids may be colored with a color contrasting against a color of the underlying body. The indicia and/or handling aids may also be the same color as the underlying prosthesis body. In some embodiments, the indicia and/or handling aids may include one or more pigments which may only be visible with special imaging tools. For example, the indicia and/or handling aids may be radiopaque, such that they may exhibit X-ray contrast relative to the wound.

In some embodiments, the indicia may include arrows, arrowheads, bullseyes. circles, and/or any other suitable shape or combination of shapes, as the present disclosure is not so limited. The indicia may indicate directionality and/or handedness (e.g., orientation) to the operator. For example, the indicia may include arrows pointing along one or more axial directions to orient the operator. In some embodiments, the indicia may be printed and/or written on the underlying prosthesis body. For example, a surgical marker may be used to provide indicia along the prosthesis. In some embodiments, a combination of ink and embroidery may be used to mark up a prosthesis. In some embodiments, the indicia may be distinguished against the prosthesis in a tactile manner, such that an operator may sense the indicia by touch and/or force feedback.

In some embodiments, the indicia and/or handling aids may be formed of a thread-like structure formed of a material similar to the underlying prosthesis (e.g., polypropylene sutures on a knitted polypropylene mesh). In other embodiments, the indicia and/or handling aids may be formed of a material different from the underlying prosthesis. It should be appreciated that combinations of material (similar to and different from the underlying prosthesis material) may be employed, as the present disclosure is not so limited.

It should be appreciated that any suitable indicia and/or handling aids located at any suitable location of a prosthesis may be employed, as the implantable prostheses of the present disclosure are not limited by the arrangement, position, distribution, number, type, shape, color, material and/or composition, bioabsorbability, and/or any other parameter of the indicia and/or handling aids. In some embodiments, an implantable prosthesis may not include any indicia.

In some embodiments, a handling aid may be formed of a strip of flexible material which may be fixed to the underlying prosthesis body at least at one point. The strip may provide similar benefits to the body as the thread-like material, namely, serving as a grab-point of the body and limiting the risk of damage or undesirable strain transferred to the prosthesis body during manipulation and handling. As will be described in further detail below, the strip may be fixed to the prosthesis body in a variety of ways to provide sufficient clearance to an operator to grasp the handling aid strip and manipulate the prosthesis body.

It should be appreciated that a prosthesis body may be formed of any suitable biocompatible material and/or combinations of materials. In some embodiments, the prosthesis body may be formed of a knitted polypropylene mesh material. It should be appreciated that the present disclosure is not limited by the material and/or structure (e.g., size, shape, arrangement) of the prosthesis body on which the handling aids are attached. As such, any suitable prosthesis body material and/or structure known in the art may be employed with the handling aids of the present disclosure.

The term “thread-like” used herein refers to any generally flexible, one-dimensional structure formed from an arrangement and/or assembly of yarns, fibers, strings, strands, threads, monofilaments, and/or multifilaments. Such structures may comprise knits, weaves, braids, non-woven materials, combinations thereof, and/or any other suitable form factor. The thread-like materials used to form one or more handling aids and/or indicia of the present disclosure may be formed of any suitable biocompatible material or combination of materials, bioabsorbable and/or non-bioabsorbable.

The term “biocompatible materials” used herein refers to materials that have the ability to perform with an appropriate host response in a specific application. Biocompatible materials have the quality of not having toxic or injurious effects on biological systems.

As used herein, the terms “bioabsorbable” or “biodegradable” refer to materials that are degraded by the body's enzymatic and/or hydrolytic pathways through a reaction against “foreign” material. Depending on the chemical nature of the material, the bioabsorbable material may disappear into the in vivo environment after a defined period, which can vary, for example, from a few hours to several months.

Examples of bioabsorbable materials include, but are not limited by, polylactic acid (PLA), polyglycolic acid (PGA), oxidized cellulose, polycaprolactone (PCL), polydioxanone (PDO), trimethylene carbonate (TMC), polyvinyl alcohol (PVA), polyhydroxyalkanoates (PHAs), polyamides, polyethers, copolymers thereof, and/or and mixtures thereof.

Examples of non-bioabsorbable materials include, but are not limited by. polyethylene terephthalate (PET), polyamides, aramids, expanded polytetrafluoroethylene, polyurethane, polyvinylidene difluoride (PVDF), polybutyl esters, polyetheretherketone (PEEK), polyolefins (such as polyethylene or polypropylene), copper alloys, silver alloys, platinum, medical grades of steel such as medical-grade stainless steel, and/or combinations thereof.

The term “implantable prosthesis” used herein refers in a non-limiting manner to a flexible plane member (e.g., a mesh or patch) of desired contour, selected in a non-limiting manner from biocompatible compositions selected from polymeric compositions; glassware; titanium containing, stainless steel, nitinol (Nickel Titanium alloys), and or other metal ware; composite materials; cardboard, natural fiber, silicone, rubber or rubber-like compositions or any mixture thereof. In some embodiments, the implantable prosthesis may be formed of polypropylene.

The term “trocar” used herein refers to a surgical instrument passed through the body or abdominal wall, used to allow easy exchange of endoscopic instruments during endoscopic or other minimally invasive surgery.

The term “hernia” used herein refers to a hernia in the abdominal cavity or in pre-peritoneal. Moreover, the term hernia may be regarded as umbilical hernia, hiatal hernia, ventral hernia, postoperative hernia, epigastric hernia, spiegelian hernia, inguinal hernia and femoral hernia, generally any abdominal wall related hernia.

It should be appreciated that the implantable prosthesis of the present disclosure may be employed in any suitable repair application, including, but not limited to, hernia repair, pelvic mesh, breast implant support, repair patch for the dura mater, inguinal hernia repair, combinations thereof, and/or any other suitable application. In some embodiments, the implantable prosthesis may be used in a repair process for a defect formed in a soft tissue.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

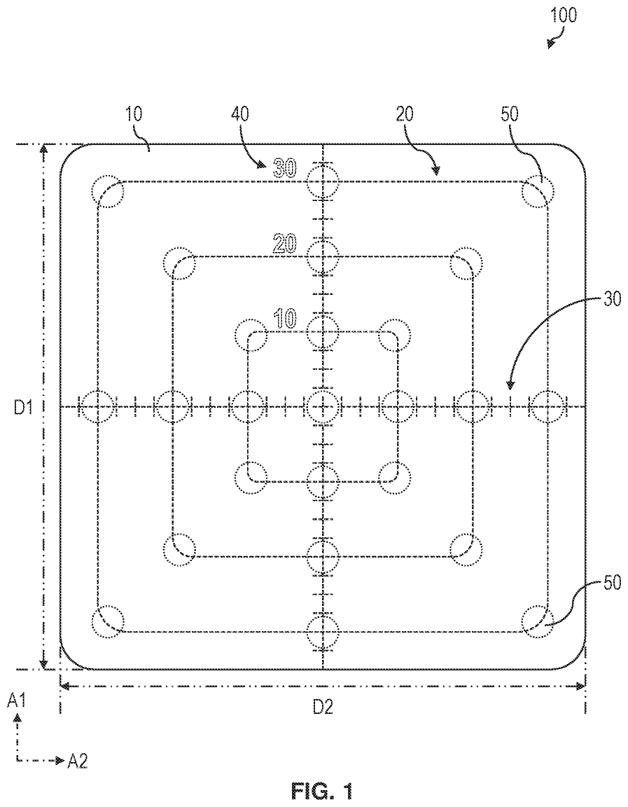

FIG. 1 shows, according to some embodiments, an implantable prosthesis 100. The prosthesis 100 may include a substantially planar body 10, which may be formed of a flexible fabric to conform to a surgical site. The prosthesis 100 may include one or more indicia to facilitate sizing, positioning, orienting, and general handling of the body 10 during operation. In some embodiments, the indicia may include axial lines 20, spatial markers 30, and/or spatial identifiers 40.

The body 10 may extend along at least two directional axes A1, A2, with at least a first dimension D1 along the first axial direction Al and a second dimension D2 along the second axial dimension A2. It should be appreciated that the body may be any suitable size for a given application and defect size. It should also be appreciated that the body may be formed (e.g., cut) into any suitable shape prior to installation, to suitably fit the intended implant location. While the body 10 is shown to be square shaped in FIG. 1, it should be appreciated that the body 10 may be any suitable shape (e.g., circle, oval, triangular) with any suitable outline shape. As such, the present disclosure is not limited by the size or shape of the prosthesis body. For example, the prosthesis body may be rectangular-shaped with a first dimension D1 of 8 cm and a second dimension D2 of 6 cm. In some embodiments, at least one dimension (e.g., dimensions D1 and/or D2) may be greater than or equal to 1 cm, 2 cm, 3cm, 4 cm, 5 cm, 6 cm, 7 cm, 8 cm, 10 cm, 12 cm, 15 cm, 20 cm, 30 cm, and/or any other suitable size. In some embodiments, at least one dimension (e.g., D1 and/or D2) of the prosthesis body may be less than or equal to 30 cm, 20 cm, 15 cm, 12 cm, 10 cm, 8 cm, 7 cm, 6 cm, 5 cm, 4 cm. 3 cm, 2 cm, 1 cm, and/or any other suitable size. Combinations of the foregoing ranges are also contemplated, including, for example, at least one dimension (e.g., D1 and/or D2) between 1 cm and 30 cm, 2 cm and 10 cm, and/or any other suitable range. Of course, other ranges, including ranges both greater than and less than those noted above are also contemplated as the present disclosure is not so limited.

In some embodiments, as shown in FIG. 1, a prosthesis body 10 may include handling aids at one or more locations 50. The handling aids may allow an operator to grasp and handle the body 10 without applying excessive stress to the body itself. The operator may manipulate the body 10 by grasping the handling aids, thereby reducing the risk of undesirable strain on the body 10 and/or tears during handling. It should be appreciated that although handling aid locations 50 are shown to follow the path of one or more indicia (e.g., axial lines 20), the handling aids may be located at any suitable position which may allow the operator to readily handle the prosthesis body.

It should therefore be appreciated that the present disclosure is not limited by the arrangement, number, and/or spacing of the handling aids. For example, although the handling aid positions are shown to be axially symmetric in FIG. 1, in some exemplary embodiments, the handling aids may be distributed non-symmetrically about axes A1, A2 of the body. In other exemplary embodiments, the handling aids may not be distributed evenly along one or more indicia (e.g., axial lines 20), in contrast to FIG. 1.

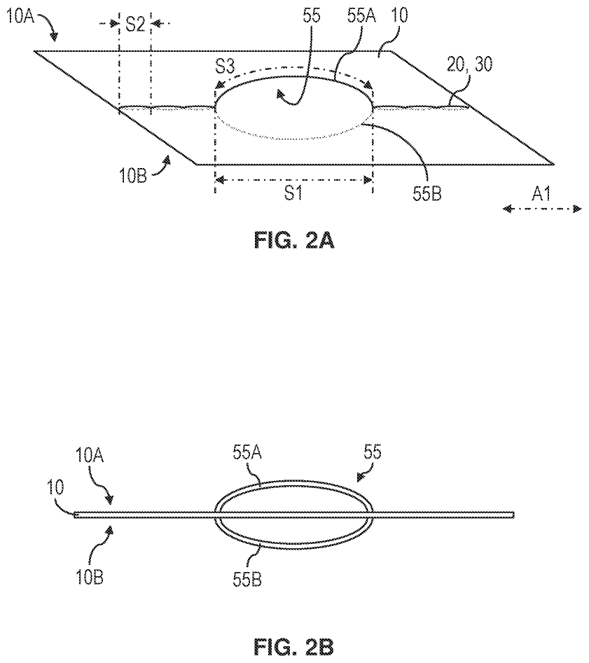

FIGS. 2A and 2B show a handling aid 55 according to some embodiments. The handling aid 55 (which may be installed in one or more handling aid positions 50, as shown in FIG. 1), may extend out of plane of a prosthesis body 10. The planar body 10 may include a first face 10A for positioning against a soft tissue surface with a defect (e.g., an abdominal wall) and a second face 10B for positioning against adjacent organs and/or soft tissue. In some embodiments, the first face 10A may include greater surface texture than the second face, such that the first face 10A may induce tissue ingrowth and accelerate healing. The second face 10B may, in some embodiments, include a barrier, such as a layer of barrier material or a barrier coating, to prevent adhesions with nearby organs and/or soft tissue.

The handling aid 55 may therefore extend from at least one face of the planar body 10. In some embodiments, the handling aid 55 may form a loop-like structure, extending out of both the first face 10A and second face 10B. In this way, the handling aid 55 may be accessible from both sides of the body 10, such that an operator may be able to grab and/or handle the prosthesis from either face. As shown in FIGS. 2A-2B, the handling aid 55 may include a first portion 55A extending out of the first face 10A of the body 10, and a second portion 55B extending out of a second face 10B of the body.

In some embodiments, the handling aid 55 may follow a line of embroidered indicia (e.g., axial lines 20, spatial markers 30), as shown in FIG. 2A. As noted previously, the handling aid 55 may be a jump-stitch in the embroidered line, such that a length S1 of the handling aid (measured along an axial direction Al of the body 10), may be greater than a length S2 of neighboring stitches. In some embodiments, the length of the handling aid may be equivalent to the length of a neighboring stitch, but the handling aid may have a span length (see length S3 in FIG. 2A) which may be greater than a span length of nearby stitches. In either embodiment, at least a portion of the handling aid 55 may be suspended above the body or otherwise unattached to the body, providing sufficient clearance in between the body 10 and the aid 55 to allow a tool to grasp said aid. In some embodiments, at least a portion of the handling aid 55 (the first portion 55A, which may be a central portion of the handling aid) may be positioned offset from a plane of the first face 10A.

It should be appreciated that the handling aid 55 may follow one or more indicia lines, as shown in FIG. 2A. In some embodiments, the positioning of the handling aid 55 along the indicia (e.g., axial lines 20, spatial markers 30) may allow an operator to readily locate the aid 55. In other embodiments, the handling aid 55 may be located independently of the indicia of the prosthesis, which may visually distinguish the handling aid from the indicia. In other embodiments still, the handling aid 55 may include separate indicia (e.g., arrows, circles, etc.) to guide an operator's eye and/or tool to the aid. It should be appreciated that the handling aids may be located at any suitable position relative to nearby indicia, as the present disclosure is not so limited. In some embodiments, a prosthesis may not include any indicia, such that the only features on the prosthesis may be the handling aids.

FIG. 2B shows a side view of a handling aid 55 installed on a prosthesis body 10. In some embodiments, the handling aid 55 may include a first portion 55A extending out of a first face 10A of the body 10 and a second portion 55B extending out of a second face 10B of the body 10. Accordingly, the handling aid 55 may be accessible from both faces of the planar body 10, such that an operator (e.g., surgeon and/or surgical tool) may grasp the handling aid 55 from either side of the body to manipulate the prosthesis.

It should be appreciated that in some embodiments, the handling aid 55 may only include one portion (e.g., portion 55A or 55B) extending out of only one face (e.g., face 10A or 10B) of the body 10. In this way, the handling aid may only be accessible from only one face of the prosthesis. In some embodiments, limiting the handling aid to only one face of the prosthesis may serve as an indicator of the orientation of the prosthesis, such that lack of access to the handling aid may indicate to the operator that the prosthesis is oriented towards one face or another. In some embodiments, a combination of handling aids positioned on one face of the prosthesis and handling aids accessible from both faces of the prosthesis may be employed, as the present disclosure is not so limited.

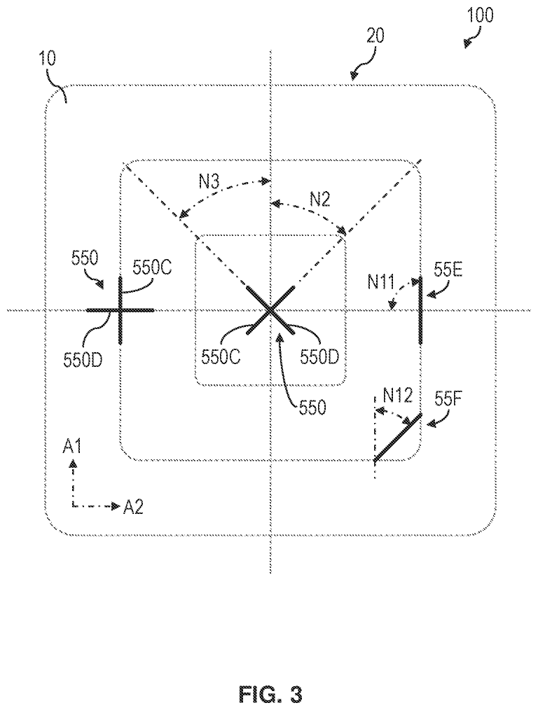

FIG. 3 shows a partial prosthesis 100 according to some embodiments. The prosthesis may be formed of a substantially planar body 10, with one or more handling aids 55E, 55F, 550 to allow the operator to handle the prosthesis. As shown in the figure, the handling aid may be oriented along an axial direction (e.g., axis A1) of the body 10. Accordingly, the handling aid may be oriented orthogonal to a secondary axis (e.g., axis A2) of the body 10. In some embodiments, the orientation of the handling aid may be determined by an angle measured between the aid and at least one axial direction of the body 10. For example, FIG. 3 shows a handling aid 55E with an angle N11 of 90° with respect to axial direction A2. In another example, FIG. 3 shows a handling aid 55F with an angle N12 of 45° with respect to axial direction A1.

It should be appreciated that in some embodiments, the handling aid may be oriented at any suitable angle with respect to the axial directions of the body. In some embodiments, the handling aid may be oriented at an angle greater than or equal to 0°, 10°,20°, 30°, 40°. 45°, 50°, 60°, 70°, 75°, 80°, 90°, and/or any other suitable angle with respect to at least one axial direction of the prosthesis. The handling aid may also be oriented at an angle less than or equal to 90°, 80°, 70°, 75°, 60°, 50°, 45°, 40°, 30°, 20°, 10°. 0°, and/or any other suitable angle. Combinations of the foregoing ranges are also contemplated, including, for example, the handling aid oriented at an angle between 0° and 90°, 0° and 45°, and/or any other suitable range. Of course, other ranges, including ranges both greater than and less than those noted above are also contemplated as the present disclosure is not so limited.

It should be appreciated that the orientation of one or more handling aids may be selected for case of manufacturing and/or to improve the function and visibility of the handling aids. In some embodiments, positioning at least one handling aid along a line of indicia may streamline the overall embroidering process of the prosthesis. In some embodiments, as noted previously, positioning the handling aid at an angle with respect to indicia may enhance the visibility of the handling aid. In some embodiments, a prosthesis may include at least one handling aid oriented in a first direction and at least one handling aid oriented in a second direction with respect to the axial directions of the prosthesis. As such, the prosthesis may include more than one handling aid positioned in a variety of different orientations to facilitate manipulation of the prosthesis with the handling aids. In some embodiments, a prosthesis may include a plurality of handling aids, all oriented in the same direction with respect to the axial directions of the prosthesis. It should therefore be appreciated that the present disclosure is not limited by the orientation of any one or combination of handling aids with respect to the axial directions of the prosthesis.

FIG. 3 also shows, according to some embodiments, a handling aid 550 with multiple legs, e.g., 550C, 550D. The additional leg(s) may improve the strength of the handling aid, such that greater tension may be applied to the handling aid while manipulating the prosthesis without risk of tearing. In some embodiments, the additional leg(s) may enhance the visibility of the handling aid. As shown in the figure, in some embodiments, the handling aids 550 may include two legs 550C, 550D, such that the handling aid may form a cross shape. In some embodiments, the legs 550C, 550D may be substantially equal in length, as shown in FIG. 3, whereas in other embodiments, each leg of a handling aid 550 may have a different length.

As discussed with respect to handling aid 55, each leg of the handling aid 550 may be oriented at an angle with respect to at least one axial direction of the body 10. For example, FIG. 3 shows a handling aid 550 with a first leg 550C oriented at an angle N2 and a second leg 550D oriented at an angle N3, with respect to an axial direction Al of the prosthesis. It should be appreciated that in some embodiments, each of the legs 550C, 550D of the handling aid 550 may be oriented at any suitable angle with respect to the axial directions of the body (e.g., axes A1 and A2 of FIG. 3). In some embodiments, at least one leg of a handling aid may be oriented at an angle greater than or equal to 0°, 10°, 20°, 30°,40°, 45°, 50°, 60°, 70°, 75°, 80°, 90°, and/or any other suitable angle with respect to at least one axial direction of the prosthesis. At least one leg of a handling aid may also be oriented at an angle less than or equal to 90°, 80°, 70°, 75°, 60°, 50°, 45°, 40°, 30°, 20°, 10°, 0°, and/or any other suitable angle. Combinations of the foregoing ranges are also contemplated, including, for example, at least one leg of the handling aid oriented at an angle between 0° and 90°, 0° and 45°, and/or any other suitable range. Of course, other ranges, including ranges both greater than and less than those noted above are also contemplated as the present disclosure is not so limited.

It should be appreciated that although legs 550C, 550D are shown to be orthogonal to one another in FIG. 3, embodiments in which the legs are not orthogonal to one another are also contemplated. In addition, although legs 550C, 550D are shown to be symmetric about the prosthesis body 10 for at least one handling aid 550 in FIG. 3, embodiments in which the legs are asymmetric about the prosthesis are also contemplated. Additionally, although legs 550C, 550D are shown to be symmetric about one another in

FIG. 3, embodiments in which the legs are asymmetric about one another are also contemplated. It should also be appreciated that although handling aids 550 are shown with a pair of legs, a handling aid according to the present disclosure may include more than two legs (e.g., three, four, five, six) legs, each at a respective angle with respect to an axial direction of the prosthesis. As noted previously, an increase in the number of legs may enhance the strength of the handling aid during manipulation.

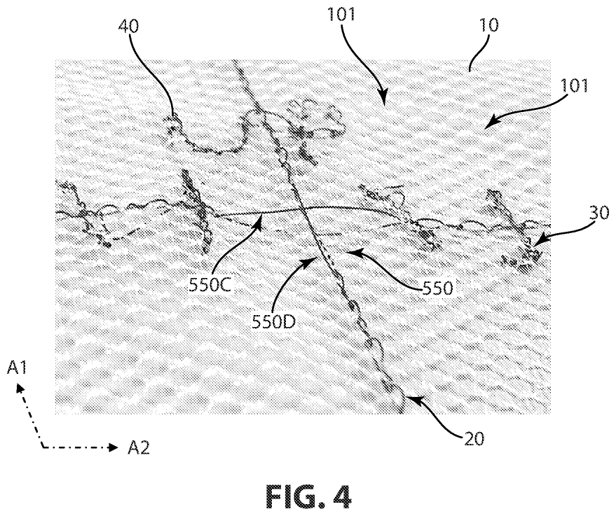

FIG. 4 shows a handling aid 550 according to some embodiments, installed on a prosthesis body 10. The handling aid 550 is distinct from the prothesis body 10. The handling aid 550 includes a first leg 550C, extending along a first axial direction Al of the body, and a second leg 550D, extending along a second axial direction A2 of the body. The handling aid 550 is shown to extend along the path of indicia such as axial lines 20. As shown in the figure, legs 550C, 550D may cross over one another to form the handling aid 550.

In some embodiments, as shown in FIG. 4, the prosthesis body 10 may have a mesh-like form factor, including one or more pores 101 distributed about its body. The inclusion of pores may significantly increase the surface area of the prosthesis, while reducing the total volume of installed material, which may be beneficial for implanted prostheses. As noted previously, the legs 550C. 550D of the handling aid 550 may span more than one pore 101, such that it may provide sufficient space for the operator to grasp and manipulate the aid.

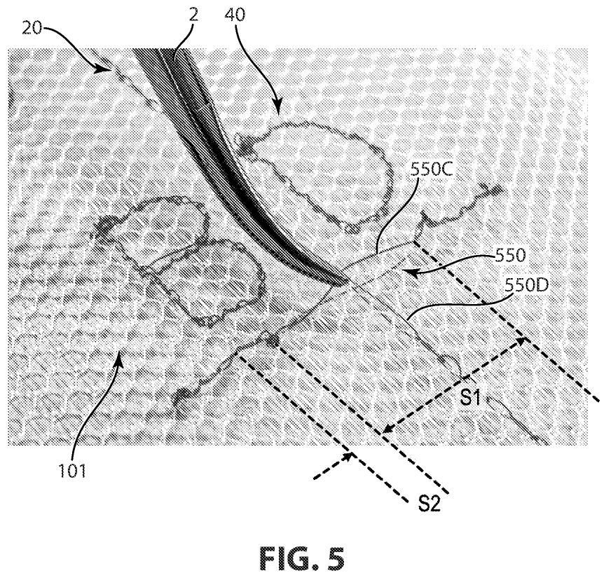

FIG. 5 shows a handling aid 550 according to some embodiments, installed on a prosthesis body 10. The handling aid 550 may include one or more legs 550C, 550D spanning more than one pore 101 of the body 10. In some embodiments, at least one leg (e.g., leg 550C) of the handling aid 550 may have a length S1, which may be greater than a length S2 of nearby stitching. As shown in the figure, a tool 2 may be used to grasp one or more legs of the handling aid 550 to manipulate the body 10. In other words, the leg length S1 may be greater than an average pore size of the body. The tool may fit within a clearance formed between the legs 550C. 550D (shown to be crossed over one another in FIG. 5) and the body 10. The handling aid 550 may be positioned in a location near an indicium (e.g., identifier 40), which may allow the operator to locate and grasp the aid 55.

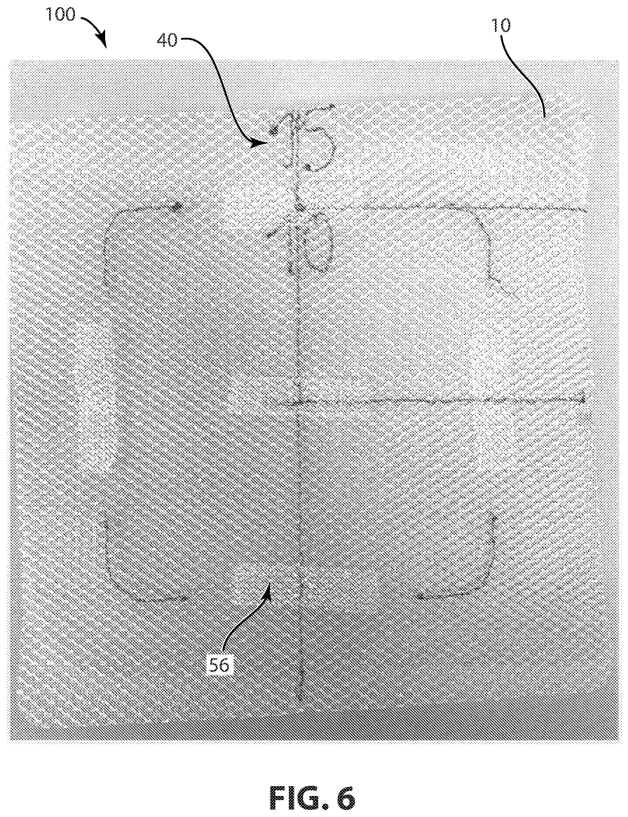

FIG. 6 shows an implantable prosthesis 100 according to some embodiments. The prosthesis 100 may include a substantially planar body 10, which may be flexible to conform to one or more soft tissue and/or organ surfaces. In embodiments represented by FIG. 6, the prosthesis 100 may include one or more handling aids 56 coupled to the body 10. In some embodiments, the handling aids 56 may be a strip of material (e.g., a mesh similar to the body 10) attached to the body 10 at two ends, such that the strip of material may serve as a handle to the body in operation.

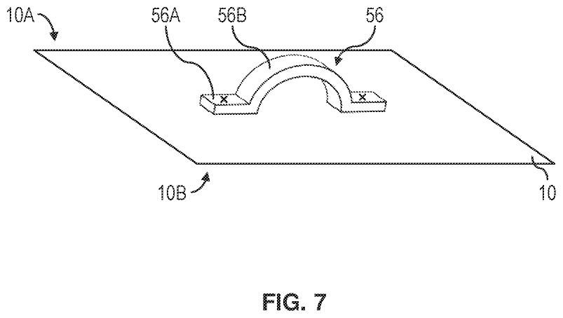

FIG. 7 shows a handling aid 56 according to some embodiments. As shown in the figure, the handling aid 56 may include one or more fixation portions 56A, which may be attached to a prosthesis body 10. In some embodiments, the fixation portions 56A may be directly sewn on the body 10, whereas in other embodiments, the fixation portions 56A may be tacked on to the body 10 with one or more adhesive materials. It should be appreciated that the handling aids 56 of the present disclosure are not limited by the means with which they may be fixed and/or attached to the prosthesis body.

The handling aid 56 may also include a free portion 56B which may be suspended between the fixation portions 56A. The free portion 56B may serve a similar purpose to the aforementioned handling aids (e.g., handling aid 55 of FIGS. 2A-2B), such that the free portion 56B may provide a clearance which the operator may grasp to manipulate the prosthesis body 10. The handling aid 56 may be formed of one or more strips of material (e.g., a rectangular strip) as shown in FIG. 7. In some embodiments, the handling aid may be formed of a flattened tube of material. The excess material may enhance the strength of the handling aid and reduce the risk of tear/breakage during handling.

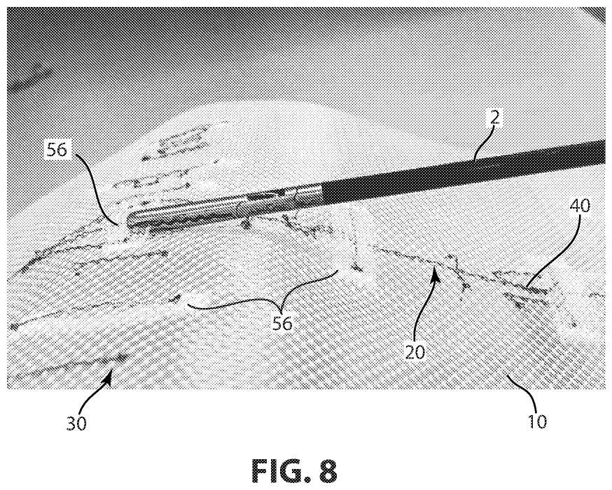

FIG. 8 shows several handling aids 56 installed on an implantable prosthesis body 10. The handing aids may be positioned along one or more indicia (e.g., spatial markers 30) of the body 10, as shown in the figure, and/or may be positioned at any suitable location irrespective of the indicia. In some embodiments, the handling aids 56 may be visually distinct on their own, due to the reduced transparency of two or more prosthesis layers compared to a single layer of the body 10. The handling aids 56 may sufficiently extend out of a plane of the body 10 to form a clearance and allow an operator and/or tool 2 to grasp the aids to manipulate the prosthesis.

It should be appreciated that the handling aids 56 shown in FIGS. 6-8 may be oriented at any suitable angle with respect to the axial directions of the prosthesis body, as discussed with reference to handling aids 55 of FIGS. 1-5. In addition, the handling aids 56 may be constructed with more than one leg (e.g., two legs crossed over one another in any suitable orientation) to enhance the strength of the handling aid 56.

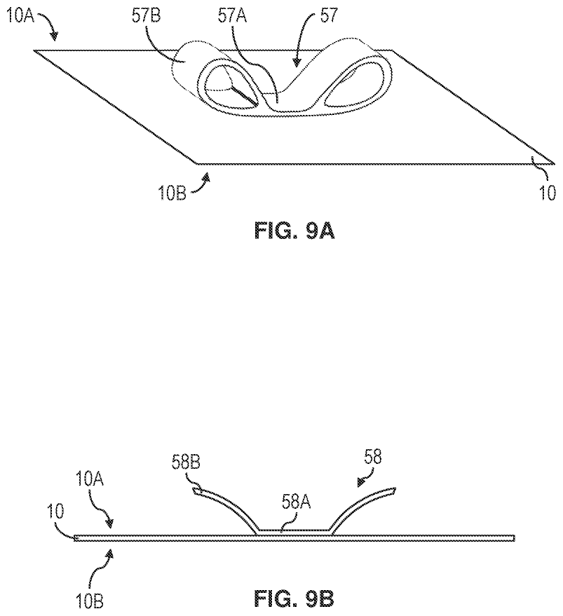

FIGS. 9A-9B show a variety of alternative embodiments of a handling aid on an implantable prosthesis body. The handling aids of FIGS. 9A-9B may be formed in a jump stitch form factor, akin to the handling aids 55 of FIGS. 1-5 and/or may be formed of a planar or strip-like material, akin to the handling aids 56 of FIGS. 6-8.

In FIG. 9A, a handling aid 57 may include one or more lobes 57B extending from the plane of the prosthesis body 10. The lobes 57B may be held in place on the body 10 with one or more fixation portions 57A. The lobes 57B may be sized to allow a surgical tool to grasp said lobe and manipulate the prosthesis. As noted previously, the current disclosure is not limited by the means with which the handling aid may be installed on the body 10. FIG. 9B represents embodiments in which a handling aid 58 includes at least one fixation portion 58A, attaching the handling aid to a prosthesis body 10. The handling aid 58 may also include one or more tabs 58B, which may serve as grab points for the operator to grasp the handling aid 58 and manipulate the prosthesis.

It should be appreciated that any of the handling aids (e.g., aids 55, 56, 57, 58) of the present disclosure may be positioned (e.g., extended out of, suspended on) on any suitable face of a prosthesis body, as the present disclosure is not so limited. The handling aids may also be positioned (e.g., extended out of, suspended on) on both faces of a substantially planar prosthesis body. In some embodiments, a combination of handling aids positioned on one face and handling aids positioned on both faces may be employed, as the present disclosure is not so limited.

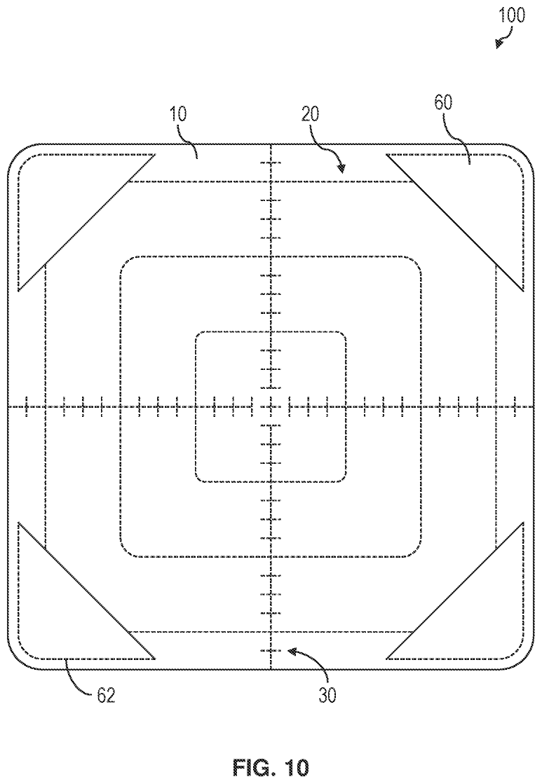

FIG. 10 shows an implantable prosthesis 100 according to some embodiments. The prosthesis 100 may include a substantially planar body 10, optionally outlined with one or more indicia (e.g., axial lines 20, spatial markers 30). The prosthesis 100 may include one or more pockets 60 formed at one or more locations of the body 10. The pockets 60 may be formed of a planar material which may be attached to the body 10 at one or more boundary lines 62. At least one side and/or portion of the perimeter of the planar material may be left open to form a pocket form factor. The pockets 60 may be attached to the body 10 in any suitable manner, such as stitching, welding, folding, and/or any other suitable technique. The pockets 60 may allow an operator to grasp the prosthesis 100 without applying undesirable strain to the underlying body 10. In some embodiments, the pockets 60 may be located at one or more corners of the body 10, as shown in FIG. 10. Of course, embodiments in which one or more pockets are located at other positions on the body are also contemplated, as the present disclosure is not so limited.

It should be appreciated that an implantable prosthesis may employ any combination of the aforementioned handling aids in any suitable orientation and arrangement on a prosthesis body, as the present disclosure is not so limited. For example, an implantable prosthesis may include one or more jump-stitch handling aids (akin to handling aids 55 of FIGS. 1-5, with one or more legs), one or more strip-like handling aids (akin to handling aids 56 of FIGS. 6-9B), and/or one or more pockets (akin to pockets 60 of FIG. 10). Any suitable combination of handling aids and pockets oriented in any suitable direction and/or extending out of one or more faces of the prosthesis body may be employed, as the present disclosure is not so limited.

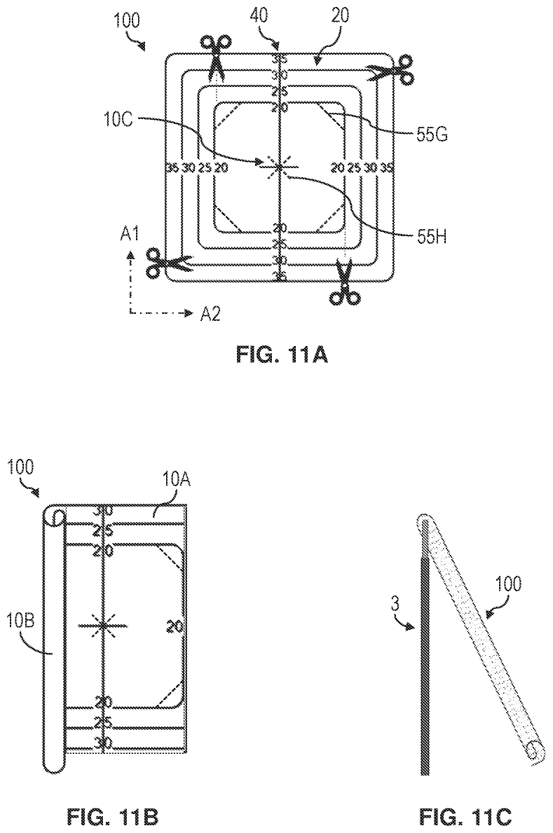

FIGS. 11A-11C show a process of preparation of a prosthesis 100 prior to installation on a defect within a subject. First, an operator may measure the effective size of a defect and determine the necessary size of the prosthesis to aid in the repair process of the defect. The size of the prosthesis may correspond to a size of the defect with margins to ensure proper coverage. The operator may subsequently select and/or cut a prosthesis meeting the size standards of the defect. As shown in FIG. 11A, the operator may cut the prosthesis along one or more indicia (e.g., axial lines 20) for ease of use. In some embodiments, the prosthesis 100 may not include any indicia, such that the prosthesis may be temporarily marked up prior to cutting (e.g., using a surgical marker).

In some embodiments, the operator may cut or otherwise form the prosthesis to maintain a degree of symmetry about at least one axial direction (e.g., directions A1, A2). The prosthesis 100 may include a central portion 10C, which may remain central to one or more axes of the prosthesis. The central portion 10C may include one or more handling aids 55H, as shown in FIG. 11A, which may be used to manipulate the prosthesis 100. The prosthesis 100 may also include one or more handling aids 55G positioned about axial markings of the body. In some embodiments, the prosthesis may include at least one aid (e.g., handling aid 55G) accessible by only one face of the prosthesis and at least one aid (e.g., handling aid 55H) accessible by both faces of a substantially planar prosthesis body.

As shown in FIG. 11B, following suitable sizing, the prosthesis 100 may be rolled such that a first face 10A may be internal to the roll, with the second face 10B external to the roll. In some embodiments, one or more indicia (e.g., spatial markers 30) may facilitate the rolling process orthogonal to at least one axial direction of the prosthesis. Subsequently, the prosthesis 100 may be grasped by a tool 3 (e.g., a 5 mm grasper), which may guide the rolled prosthesis 100 into a trocar inserted near a surgical site.

Once the prosthesis has been inserted into the surgical site, it may be unrolled such that an external face of the prosthesis (e.g., face 10B of FIG. 11B) may be facing the defect. As noted previously, handling aids 55G may be accessible from at least the external face of the prosthesis. Accordingly, such handling aids may assist a surgical tool in the unrolling, smoothing, and/or positioning of the prosthesis with respect to the defect. The surgical tool may subsequently center the prosthesis with respect to the defect using one or more handling aids (e.g., aid 55H shown in FIG. 11A) positioned centrally to the prosthesis (e.g., located at central portion 10C shown in FIG. 11A). The central handling aids may be accessible from both sides of the prosthesis and may help align the prosthesis with the defect. Once the prosthesis has been determined to be installed appropriately (e.g., with visual inspection), the prosthesis may be secured to the defect site with any suitable means (e.g., sutures, staples, tacks, combinations thereof, etc.). Although an exemplary repair process with a prosthesis is described herein, it should be appreciated that the prostheses of the present disclosure are not limited by the techniques with which they may be delivered, installed and/or secured to the defect site.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter.

What is claimed is:

1. A surgical prosthesis comprising:
a mesh, the mesh comprising a first face and a second face; and
a loop distinct from the mesh, the loop being attached to at least two locations on the mesh, the loop having a central portion between the at least two locations, the central portion being unattached to the mesh and disposed on a first side of the first face.

2. The surgical prosthesis of claim 1, further comprising at least one stitch line with an average stitch length greater than an average pore size of the mesh, wherein the loop has a span length greater than the average stitch length.

3. The surgical prosthesis of claim 1, wherein the central portion is offset from a plane of the first face.

4. The surgical prosthesis of claim 1, further comprising a secondary central portion of the loop, wherein the secondary central portion of the loop is accessible from the second face, and wherein the central portion of the loop is accessible from the first face.

5. The surgical prosthesis of claim 1, further comprising a mesh strip attached to the mesh at least at one location, wherein at least a portion of the mesh strip is offset from the first face.

6. The surgical prosthesis of claim 5, wherein the mesh strip is formed of a collapsed tube.

7. The surgical prosthesis of claim 1, further comprising at least one stitch line visually distinct from the mesh.

8. The surgical prosthesis of claim 7, wherein the at least one stitch line is colored differently from the mesh.

9. The surgical prosthesis of claim 1, wherein the loop is aligned with an axial direction of the mesh.

10. The surgical prosthesis of claim 3, wherein a clearance in between the first face and the loop is configured to fit at least a portion of a surgical tool.

11. The surgical prosthesis of claim 1, further comprising a secondary loop oriented at an angle relative to the loop.

12. The surgical prosthesis of claim 1, wherein the loop is disposed central to the mesh.

13. The surgical prosthesis of claim 1, wherein the loop is disposed proximal to a periphery of the mesh.

14. The surgical prosthesis of claim 1, wherein the loop and the mesh are formed of the same material.

15. A method of handling a surgical prosthesis comprising:
grasping a central portion of a first loop, the first loop being attached to two locations on a mesh of the surgical prosthesis, the mesh comprising a first face and a second face, the first loop being distinct from the mesh; and
manipulating a position of the central portion with respect to a defect in a patient.

16. The method of claim 15, further comprising rolling the mesh, and wherein grasping the central portion of the first loop unrolls the mesh.

17. The method of claim 15, further comprising:
rolling the mesh; and
grasping a central portion of a second loop to unroll the mesh.

18. The method of claim 15, further comprising grasping a central portion of a second loop from the second face of the mesh.

19. The method of claim 15, further comprising grasping the central portion of the first loop from the first face of the mesh. 20 The method of claim 19, further comprising grasping a secondary central portion of the first loop from the second face of the mesh.

21. The method of claim 15, further comprising attaching the mesh around the defect.

22. The method of claim 15, further comprising grasping a mesh strip attached to the mesh at least at one location, wherein at least a portion of the mesh strip is offset from the first face.

23. The method of claim 22, wherein the mesh strip is formed of a collapsed tube.

24. The method of claim 15, further comprising grasping the central portion of the first loop with a surgical tool.

25. The method of claim 15, further comprising grasping a central portion of a second loop, the second loop attached to the mesh at least at two locations, the second loop oriented at an angle relative to the first loop.

26. The method of claim 15, further comprising grasping at least one pocket of the mesh to manipulate the mesh relative to the defect, wherein the at least one pocket is attached to the mesh at least at one boundary.
